(19) United States
(12) Patent Application Publication
(10) Pub. No.: US 2015/0185198 A1
(43) Pub. Date: Jul. 2, 2015

(54) METHOD AND SYSTEM FOR QUANTIFYING REAL TIME ION CHANNEL ACTIVITY WITH STANDARDIZED VALUES

(71) Applicants: Peeyush Shrivastava, Columbus, OH (US); Vineet Erasala, Mason, OH (US)

(72) Inventors: Peeyush Shrivastava, Columbus, OH (US); Vineet Erasala, Mason, OH (US)

(21) Appl. No.: 14/144,428

(22) Filed: Dec. 30, 2013

(51) Int. Cl. G01N 33/48

(52) U.S. Cl. CPC G01N 33/48

(57) ABSTRACT

A method and system through which input electrophysiological data from a patient or model organism can be integrated within a computational backend environment where the input parameters are combined to quantify and output voltage-gated channel activity having picoamperes/second units. The computational backend environment then diagnoses membrane excitability conditions based on the comparison of the channel activity quantification to a theoretical database of channel activity quantifications. In this regard, the computational backend environment can quantify electrophysiological data in a way which allows for an accurate diagnosis and output of treatment options based on the quantification of several ion channels.

### BACKGROUND OF THE INVENTION

1. Field of the Invention

This invention relates generally to measuring and analyzing ion channel activity and, more particularly, to a method and system for quantifying measurements of activity from a plurality of different ion channels with standardized values having a real time context, enabling the entire electrophysiology of an organism to be mapped and evaluated.

2. Description of the Prior Art

Ion channels are embedded in the plasma membranes of all cells and are well known as pore-forming membrane proteins which perform a substantial number of vital functions for the cell on which it is present. While the study of ion channels is relatively new, ion channels are now known to be integral components of the nervous system of living organisms as well as components in a many biological processes involving rapid changes in cells. There are over 300 types of ion channels; but when classified by the type of ions, ion channels may be categorized as chloride channels, potassium channels, sodium channels, calcium channels, proton channels, or non-selective cation channels.

Irregularities or other disruptions in the function of ion channels in an organism are known to be associated with disease or dysfunction in the organism. Indeed, the association between irregularities in particular ion channels and certain disorders or diseases is well known. Such ion-based diseases include cystic fibrosis, long QT syndrome, and Brugada syndrome, and typically have devastating consequences for the organism afflicted therewith. Providing treatment for ion-based diseases customarily involves protocols which are designed to address an irregularity in the particular ion channel known to be associated with the targeted disease.

Thus, diagnosing the irregularities which cause ion based diseases, as well as the development of treatment protocols therefor, necessarily depends on the ability to measure activity of ion channels. Patch clamping is a common technique for measuring the activity of a single or multiple ion channels to enable it to be quantified and studied. In this regard, conventional medical care practices are often based on utilizing measurements for activity of a particular, targeted ion channel, which have been obtained through patch clamping, to identify irregularities in the activity of the targeted ion channel and develop treatment protocols directed at specific molecular mechanisms in an attempt to rectify the identified irregularity.

While the ability to identify irregularities in ion channel activity, diagnose diseases associated with such irregularities, and develop treatment protocols designed to address the irregularity represents a substantial improvement in the available medical care for a patient afflicted with a ion-based disease, a problem which still exists is the conventional diagnoses and treatments for ion-based disease typically only take into account information concerning the ion channel associated with a particular disease sought to be treated. For example, if cystic fibrosis is based on irregularities within the chloride ion channel, conventional diagnoses are based solely on the activity of the chloride channel, with the activity of the other ion channels are ignored. Such an approach is inherently limited, however, because all treatment protocols are likely to affect the activity of not just the targeted ion channel, but of a plurality of ion channels. Moreover, the differences in the activity of a patient's other ion channels contribute to one person's cystic fibrosis being completely different than another person's; and may necessitate a completely different treatment protocol based on how it will affect the activity of all of the patient's ion channels. Consequently, if there is no consideration for the ion flux in each ion channel in a patient, as well as how such ion flux will be affected by a particular treatment, the same treatment protocol which may be helpful for one patient may not provide optimal-results for another patient.

A related problem in the conventional diagnoses and treatment of ion-based diseases is that the current technique to quantify channel activity for a certain ion channel lacks both standardization (and units of measurement) and real time parameters. Because of this lack of standardization, it is impossible under conventional methods to analyze the entire human electrophysiology with any existing perspective. And because current techniques lack real time parameters, the ability to map such electrophysiology data with time frame parameters vital for the most accurate diagnosis is non-existent.

In order to more accurately predict, as well as to interpret, a patient's response to treatments for ion based diseases, such as excitable cell drugs, the patient's entire excitable cell physiology must be measured, standardized, mapped, and compared to desirable baseline values. Thus, there remains a need for a method and system which enables measured activity of a plurality of ion channels to be quantified with standardized values and given a real time context. It would be helpful if such a method and system for quantifying ion channel activity was able to summarize the activity of four key voltage gated ion channels for each excitable cell type (cardiac, pancreatic, beta, neuron, nephron etc.). It would be additionally desirable for such a method and system to facilitate comparisons of such summaries obtained from a given patient over a representative period of time to a database compiled using theoretical healthy channel activities for each organ, thus enabling a diagnosis to be configured based on the entire excitable cell physiology of the patient. It would further be desirable if such a method and system was embodied in a device which not only processed data from each excitable cell type, but also integrated flow cytometry, electrophysiology, and cell-cell differentiation to actually obtain data streams from each excitable cell type to be processed.

The Applicant's invention described herein provides for a method and system adapted to allow channel activity over a plurality of ion channels to be quantified with standardized values and given a real time context to enable the results to be compared between ion channels within a given patient or with normal human electrophysiology (or normals) for a particular ion channel. The primary steps of Applicant's method for quantifying measurements of activity from a plurality of different ion channels include measuring real time data from a plurality of ion channels, generating standardized values from the real time ion channel data, and summarizing the activity of four key voltage gated ion channels for each excitable cell type from the standardized values. When in operation, the method and system allows diagnoses of ion based diseases to include, and recommended or possible treatment protocols for ion based diseases to be configured with, standardized, real time data from a patient's entire excitable cell physiology. As a result, many of the limitations imposed by the prior art are removed.

### SUMMARY OF THE INVENTION

A method and system through which input electrophysiological data from a patient or model organism can be integrated within a computational backend environment where the input parameters are combined to quantify and output voltage-gated channel activity having picoamperes/second units. The computational backend environment then diagnoses membrane excitability conditions based on the comparison of the channel activity quantification to a theoretical database of channel activity quantifications. In this regard, the computational backend environment can quantify electrophysiological data in a way which allows for an accurate diagnosis and output of treatment options based on the quantification of several ion channels.

The software implemented method and system comprises the primary steps of providing measurements of a subject's ion channel activity for a plurality of voltage gated ion channels, processing the measurements of ion channel activity, and interpreting the processed measurements of ion channel activity. The measurements of ion channel activity of a subject include measurements representing the number of active channels for each ion and measurements of the rate of flow of ions for each channel for a representative time frame. Processing the measurements of ion channel activity results in the calculation of a standardized value for channel activity from the number of active channels for each ion and data related to the rate of flow of ions for four specified key voltage gated ion channels: sodium, calcium, potassium, and chloride, for each excitable cell type measured. Interpreting the processed measurements of ion channel activity is performed through mapping of the entire electrophysiology of a subject to enable comparisons to the electrophysiology for both known disorders and healthy baselines, as well as consideration in the diagnosis and development of treatment protocols of how each ion channel will be affected by the protocol. In addition, prior to processing, the software implemented method and system may also perform a step of configuring electronic files containing the measurements of ion channel activity, if such files are in a non-readable file format, to enable the underlying the measurements to be extracted and populated into readable file formats.

It is an object of this invention to provide a method and system which enables measured activity of a plurality of ion channels to be quantified with standardized values and given a real time context.

It is another object of this invention to provide a method and system for quantifying ion channel activity which is able to summarize the activity of four key voltage gated ion channels for each excitable cell type.

It is yet another object of this invention to provide a method and system which compares such summaries obtained from a given patient over a representative period of time to a database compiled using theoretical healthy channel activities for each organ, thus enabling a diagnosis to be configured based on the entire excitable cell physiology of the patient.

It is still another object of this invention to provide a method and system embodied in a device which not only processed data from each excitable cell type, but also integrated flow cytometry, electrophysiology, and cell-cell differentiation to actually obtain data streams from each excitable cell type to be processed

These and other objects will be apparent to one of skill in the art.

### BRIEF DESCRIPTION OF THE DRAWINGS

FIG. 1 shows a general process flow according to the present invention.

FIG. 2 shows a data quantification process in accordance with the present invention.

FIG. 3a is a block diagram of representative data values and calculations for data collection for conventional ion channel activity calculations.

FIG. 3b depicts an equation for channel activity for conventional ion channel activity calculations.

FIG. 4 is a block diagram of representative data values and calculations for data collection for ion channel activity calculations in accordance with the present invention.

FIG. 5 depicts a exemplary ion channel data result mapping in a data handling system built in accordance with the present invention.

FIG. 6 is a block diagram showing the processes of an apparatus adapted to allow channel activity over a plurality of ion channels to be quantified with standardized values and given a real time to enable the results to be compared between ion channels within a given patient or with theoretical normals for a particular ion channel.

FIG. 7 is a block diagram showing the processes of a cloud based implementation for quantifying channel activity over a plurality of ion channels with standardized values in real time, enabling the results to be compared between ion channels within a given patient or with theoretical normals for a particular ion channel.

FIG. 8 shows a data configuration process in accordance with the present invention.

### DETAILED DESCRIPTION OF THE INVENTION

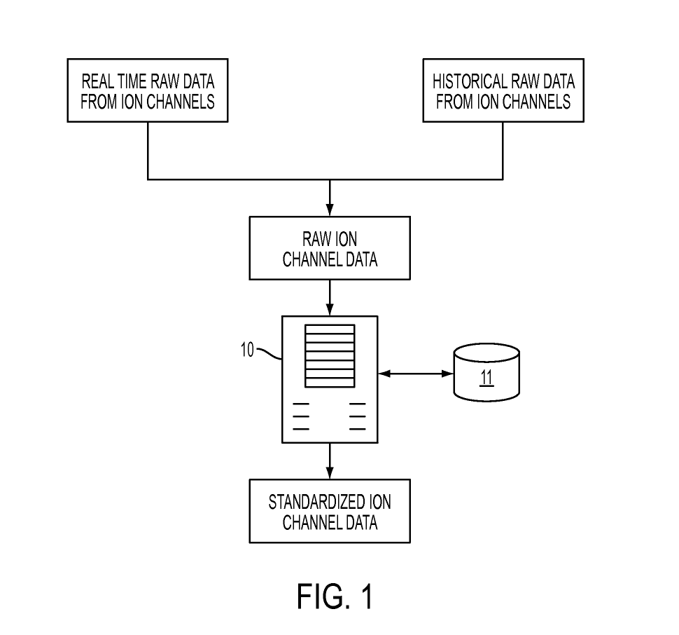

Referring now to the drawings and in particular FIG. 1, an overview of the general process flow of a method for quantifying measurements of activity from a plurality of different ion channels is shown. A data handling software application, embodied as computer software containing instructions which cause a processor of an electronic device having computing capability to perform the necessary steps to quantify electronic files containing ion channel activity measurements embodied as ion channel activity data, is loaded onto a data handling system 10 to perform the principle operational steps of the ion channel activity measurement quantification. The data handling system 10 is defined as a device having computing capability having the data handling software application installed thereon, and may be embodied as a network connected server or computer which receives ion channel activity data remotely from client software or over a bus or network interface, or as an electronic device with computing capability which captures or is provided such measurements locally. The data handling system 10 operates in conjunction with an activity database 11 from which it can access stored data points relating to ion activity measurements of normals (theoretical or observed), known diseases, and past quantifications. In addition, the activity database 11 provides a location in which ion channel activity data processed by the data handling system 10 may be catalogued for future use.

Measured (or raw) ion channel activity data is defined as electronic files containing measurements of ion channel activity at a given time or over a defined time period. As such, measured ion channel activity data includes measurements relating to the number of active channels for four specified key voltage gated ion channels, sodium, calcium, potassium, and chloride, for each excitable cell type measured, as well as the flow of ions through the channels during the course of a predetermined, representative time frame. The measured ion channel activity data may include either measured ion channel activity data which is being captured at or around the time of quantification, measured ion channel activity data which from historical captures which has been stored, or both. This measured ion channel activity data is provided to the data handling software application to be processed and output as processed ion channel data having standardized values with time perspective. Through this processing, channel activity measurements are quantified in a manner which enables comparisons of values between ion channels within a given patient or with baseline values (including normals, whether theoretical of from analysis of past quantifications, known diseases, and historical quantifications) for a particular ion channel.

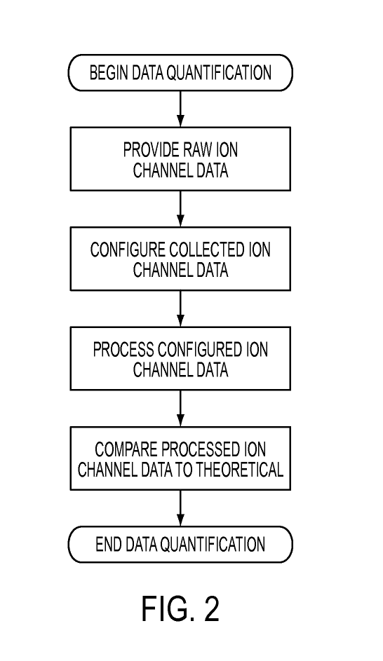

Referring now to FIG. 2, the quantification process for measured ion channel activity data for a patient begins with the provision of measurements of ion channel activity for each of the key voltage gated ion channels for the various excitable cell types as well as the ion flow through the channels over a period of time to a data handling software application in an electronic file. It is contemplated that such measurements may also be manually entered into a data handling system to be stored in an electronic file. The measurements may be provided through the collection of measurements at or around the time of quantification on a device connected to a data handling system, or by uploading such measurements from a database or a remote collection or storage device. It is contemplated that this incoming data may be embedded in different file formats depending on the specification of either the measurement apparatus or device inputting such data. Consequently, once the measured ion channel activity data is provided, the ion channel activity data is first configured by analyzing it and, if necessary, converting to or extracting it into an electronic file in a data format which can be read and processed by the data handling software application.

After the measured ion channel activity data has been configured, it is processed by the data handling software application to produce quantified measurements of ion channel activity. In this regard, processed (or quantified) ion channel activity data is defined as an electronic file having quantified measurements, providing standardized values having known units and enabling standardization across different ion channels and across different excitable cell types. The data handling software application is then configured to provide for comparisons of quantified measurements, through which the processed ion channel activity data is analyzed with other standardized measurement values, such as theoretical normals, providing perspective to the standardized values of the processed ion channel activity data across the entire electrophysiology of the patient from which the measured ion channel activity data was obtained.

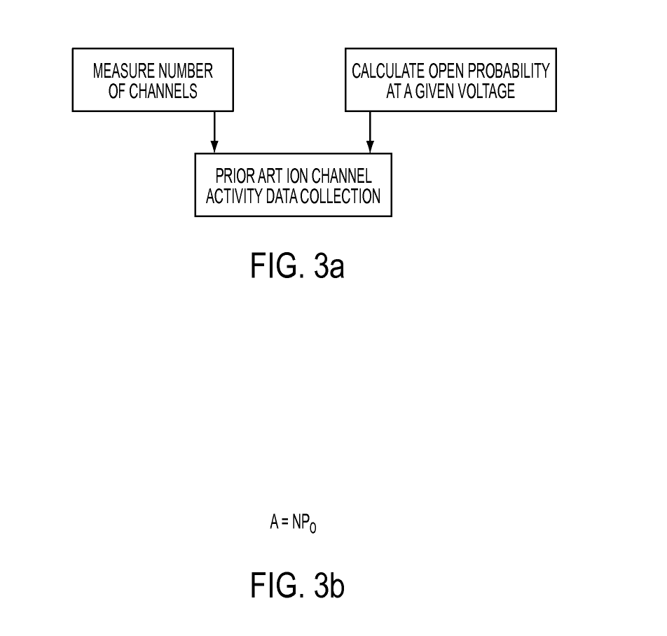

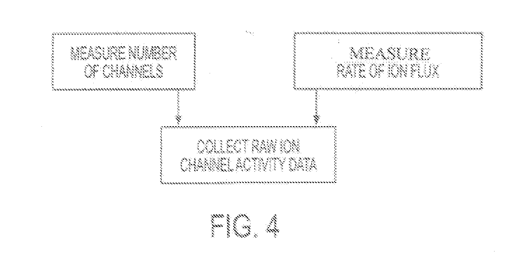

Referring now to FIGS. 3a and 3b, data collection for calculating channel activity (“A”) for an excitable cell type conventionally characterized in terms of two key parameters; the number of active channels (“N”) and the open probability of a voltage gated ion channel (“Pₒ”). The number of active channels can be measured directly using an electrophysiological technique such as a patch clamp technique. The open probability is typically calculated indirectly, through the calculation of the fraction of time a patch clamp reveals a single ion channel is open versus closed. But a clear limitation of the use of these parameters, however, is that the channel activity calculation produces a result which lacks units and thus cannot be standardized. As such, this characterization of channel activity produces a result which does not enable effective comparisons, which is unable to provide substantial time context, and which does not provide context to enable the entire excitable cell physiology to be mapped. Accordingly, the quantification of real time ion channel activity (or ion channel activity over a historical period of time) with standardized values is not accomplished through the use of the conventional parameters of ion channel activity data collected.

Referring now to FIG. 4, the measurements be used by the data handling software application to produce standardized, unit based ion activity calculations are obtained by running a patch clamp technique to measure the number of active channels while running a patch clamp technique to measure the rate of flow of ions. The rate of flow is determined by calculating the quotient of a measured flow of ions over the course of a period of time and the period of time. In the preferred embodiment, the representative period of time utilized is 120 seconds.

By augmenting the conventional data collection in such a manner, measured ion channel activity data which enables the data handling software application to calculate a channel activity value for an excitable cell to be quantified with known, easy to interpret units and with a time component can be collected. This is done by calculating channel activity from the number of active channels and the rate of ionic flux for any particular ion. Through the utilization of the measured rate of ionic flux for any particular ion as opposed to an open probability, a channel activity value is generated which can be expressed in picoamperes/second and which is calculated using either a real time data feed or historical data. Because channel activity is expressed with these standard units of measurement, channel activity values calculated by the data handling software application from this raw ion channel activity data can be compared, in context, with other channel activity values across different ion channels and across different excitable cell types.

The use of the number of active channels and the rate of ionic flux for any particular ion provides a key extension of the equation of FIG. 3b in that it more substantially maintains the foundations of electrical physics. In electrical physics, electrical work is well known to be the product of electrical potential and the change in magnitude of charge. If ion flux is considered as a system of analysis, with ionic movement being a representation of the capacity of said system to perform electrical work, then the equation of FIG. 3b is verified with the open probability encapsulating both parameters of electrical work. To explain, the open probability of a voltage gated ion channel is dependent on the membrane potential of the cell observed at any given time. Thus, the open probability is a dynamic value, and influences the magnitude of ion flux occurring at any given moment. But by using the number of active channels and the rate of ionic flux for any particular ion, real time data is able to be processed and results are expressed in terms of electrical current/time. Furthermore, this not only encompasses the open probability of the channel, it integrates it with the change in the magnitude of the charge wholly lacking from the equation of FIG. 3b.

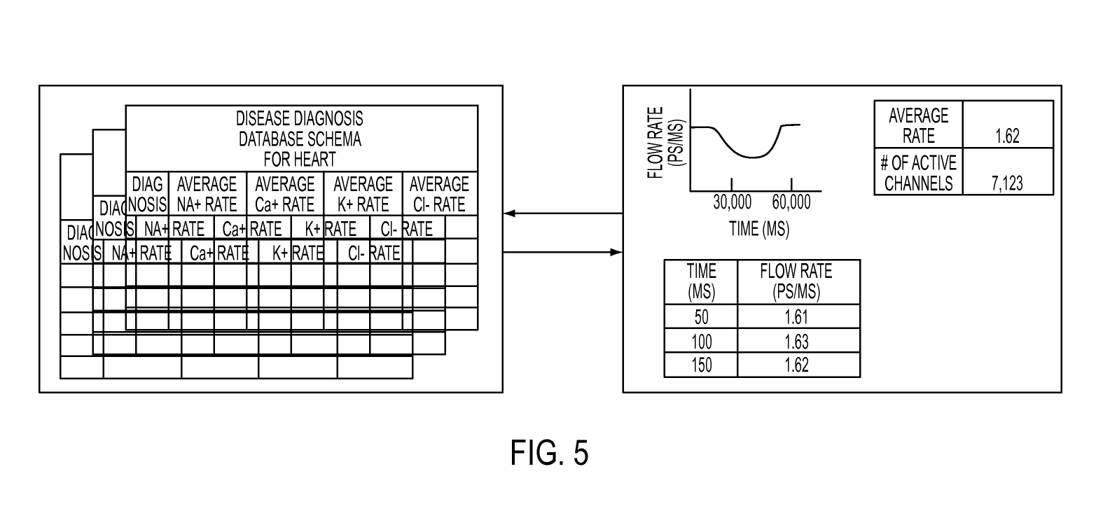

In the preferred embodiment, the data handling software application ion channel activity data is processed in SQL backend, where the incoming parameters of the number of active channels and the rate of ionic flux for any particular ion are used to produce several outputs for channel activity. Referring now to FIG. 5, the outputs include a graphical display of how ion fluxes change over a representative recording time, a calculation of the average channel activity for multiple ion channels in multiple membrane excitability disorders, as well as a table of the changing values per a constant 50 millisecond interval. In this regard, activity measurement related data which is provided in real time as well as activity measurement related data and baseline related data which is stored on an activity database is integrated to provide outputs which may be utilized in diagnosis and treatment of known disorders.

It is contemplated that the activity database is initially populated with several thousand theoretical channel activity data points processed in accordance with the present method. It is further contemplated that as channel activity data points are generated through the processing of measured channel activity, and as additional ion based diseases are discovered and channel activity data points are measured and associated therewith, such measured channel activity data points and channel activity data points for membrane excitability disorders may be added to the activity database.

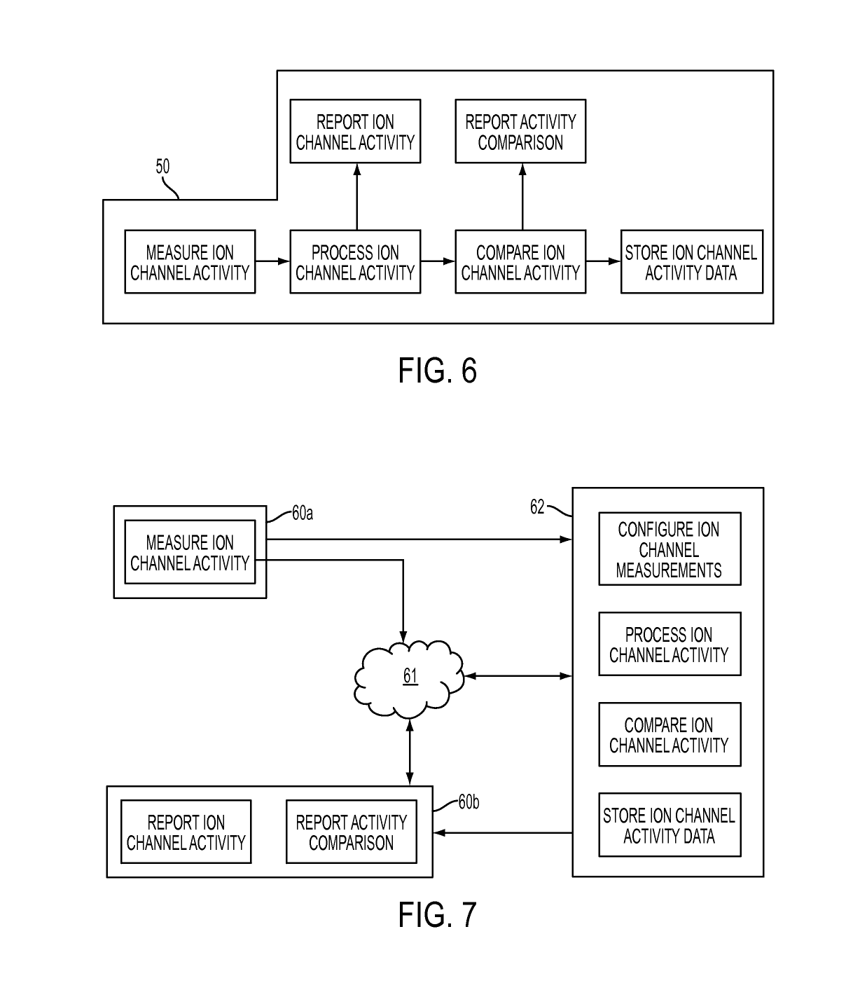

Referring now to FIG. 6, in one embodiment, the data handling software application may be on an integrated device 50 which measures the requisite ion channel activity values and outputs the standardized channel activity and/or a diagnosis in addition to performing the processing and analysis on an integrated display. In such an embodiment, the integrated device 50 is configured to perform the requisite patch clamping techniques, process the ion channel activity values, perform the comparison based on an analysis of standardized values taken in real time and the average channel activity for ion channels in membrane excitability disorders to render a diagnosis or assist in the development of a treatment protocol, and generate a report containing a summary, diagnosis, and/or treatment recommendations.

In any embodiment, a report on ion channel activity typically includes the calculated channel activity value of each measured voltage gated channel, and may include the number of active channels and information relating to the rate of ionic flux for any particular ion. A report on activity comparison provides diagnosis related information generated from the comparison of ion channel activity, in that standardized values for the different measured ion channels are compared and contrasted with standardized baseline values. Considering the entire electrophysiology of the patient, a parallel set of baseline values is sought and then matched with the patient. A diagnosis and treatment protocol is based on the diagnosis and optimal treatment for the matched baseline value. As such, it is contemplated that an activity database built in accordance with the present invention will additionally include suggested or ideal treatment protocols for known diseases. It is additionally contemplated that information related to lifestyle factors, and how they affect channel activity, may be included into the database as lifestyle factor data. In this regard, information related to the lifestyle of a patient seeking analysis of ion channel activity may be considered in the generation of a report. It is contemplated that such lifestyle factors would include dietary habits, exercise, stress levels, and chemical exposure among a host of others.

Referring now to FIG. 7, in another embodiment, a discrete measuring device 60a is employed to measure the requisite ion channel activity values and a discrete display device 60b is employed to output the standardized channel activity and/or a diagnosis. In this embodiment, substantially the same processes are performed as when performed on an integrated device, but through a plurality of discrete parts sharing a data connection. The measuring device 60a is connected to a computer network 61, such as the Internet, through a wired connection or a wireless connection, and configured to transmit data to a processing device 62. In the alternative, the measuring device 60a may be directly connected to the processing device 62, again through a wired or wireless connection. In either case, the measure device 60a is configured to transmit measured ion channel activity values to the processing device 62, for configuration, processing, and comparison to existing values. Once the processing device 62 performs its functions, the results are transmitted to the display device 60b, either through a wired or wireless connection to the computer network 61 or through a wired or wireless direct data connection. While an implementation with a discrete measuring device 60a and display device 60b is shown, it is contemplated that a similar implementation wherein the measuring device is integrated with the display device may be employed as well. In such an implementation, the integrated measuring and display device would still communicate data with the processing device through a wired or wireless data connection.

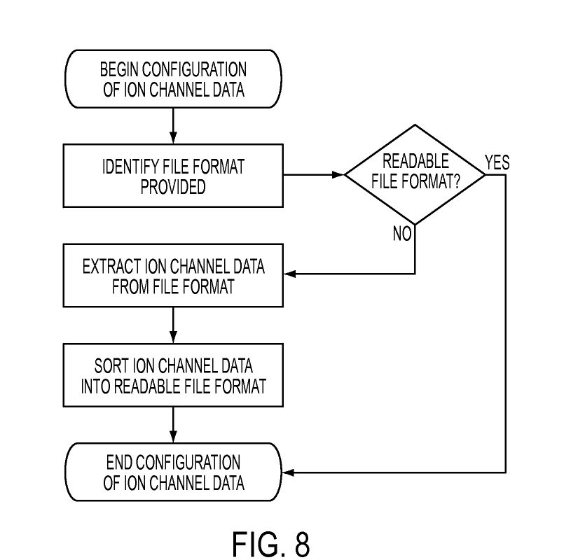

Referring now to FIG. 8, because it is understood that in some embodiments, measured ion channel activity data may be provided in various file formats, the data handling software application is adapted configure ion channel activity data received from a discrete device. The configuration begins with checking the file format of ion channel activity data received from a remote electronic device. Then, the data handling software application utilizes embedded emulators which are adapted to temporarily simulate the runtime environment of the file format common in healthcare informatics. These emulators enable the data handling software to generate virtual executables and provide the data handling software application with plug and play functionality. In the preferred embodiment, the data handling software application is configured to either directly read or extract data through the use of emulators from file formats such as CSV, HL7, SQL, Oracle RDBMS, or formats configured to be accessible through IOS, Android, Windows, Unix, and Linux operating environments.

Accordingly, the configuration process of ion channel data begins with identifying the file format of data provided. If the file format contains measurement values which are directly readable, the configuration process is not necessary and the data handling software application terminates the configuration process of ion channel data. If the file format does not contain directly readable measurement values, the emulator associated with the file format identified is run to enable the requisite measurements from the ion channel data extracted from the file format and populated in a directly readable file format. Once the requisite measurements for ion channel data are populated into a readable file format, the data handling software application terminates the configuration process of ion channel data.

The instant invention has been shown and described herein in what is considered to be the most practical and preferred embodiment. It is recognized, however, that departures may be made therefrom within the scope of the invention and that obvious modifications will occur to a person skilled in the art.

1. A method for quantifying voltage gated channel activity for a subject, comprising the steps of:
providing an electronic device having at least one processor configured to execute instructions from a software application;
providing ion channel activity data for a plurality of predetermined voltage gated ion channels, wherein said ion channel activity data defines measurements taken in a patient over a specified time period and includes, for at least one given excitable cell type, a measurement of the number of channels for each voltage gated ion channel and measurements of the ionic flux for each voltage gated ion channel during the specified time period; and
processing by said processor the ion channel activity data provided, wherein the step of processing includes (1) calculating an ion channel activity value for each of the plurality of voltage gated ion channels from the number of channels measurement and the ionic flux measurements over the specified time period and (2) producing therefrom a quantified provided ion channel activity value for each voltage gated ion channel over the given excitable cell type that is standardized for expression in electric current by time and voltage units, thereby enabling real time activity of the patient's voltage gated ion channels for the entire given excitable cell type to be summarized and real time ion channel activity values calculated for distinct ion channels and/or across different excitable cell types to be compared.

2. The method for quantifying voltage gated channel activity for a subject of claim 1, additionally comprising the steps of:
providing an activity database having at least one quantified baseline ion channel activity value; and
interpreting by said processor quantified provided ion channel activity values, wherein the step of interpreting includes comparing the quantified provided ion channel activity values with quantified baseline ion channel values.

3. The method for quantifying voltage gated channel activity for a subject of claim 2, wherein said quantified baseline ion channel activity values include theoretical ion channel activity values for at least one organ or cell type.

4. The method for quantifying voltage gated channel activity for a subject of claim 2, wherein said quantified baseline ion channel activity values include historical ion channel activity values.

5. The method for quantifying voltage gated channel activity for a subject of claim 2, wherein said quantified baseline ion channel activity values include known disease ion channel activity values for at least one organ or cell type.

6. The method for quantifying voltage gated channel activity for a subject of claim 5, wherein the step of interpreting additionally includes diagnosing membrane excitability disorders present by identifying known disease ion channel activity values which are substantially similar quantified provided ion channel activity values.

7. The method for quantifying voltage gated channel activity for a subject of claim 6, wherein the activity database additionally includes lifestyle factor data associated with ion channel activity and the diagnosing of membrane excitability disorders present includes a comparison of lifestyle factor data lifestyle factors relevant to the subject.

8. The method for quantifying voltage gated channel activity for a subject of claim 1, wherein the step of processing includes calculating ion channel activity in real time.

9. The method for quantifying voltage gated channel activity for a subject of claim 1, additionally comprising the step of outputting by said processor ion channel activity, wherein the step of outputting includes providing a visual display of quantified provided ion channel activity values.

10. The method for quantifying voltage gated channel activity for a subject of claim 1, additionally comprising the step of configuring ion channel activity data provided by said processor, wherein the step of configuring includes determining the file format of the ion channel activity data provided.

11. A system for quantifying voltage gated channel activity for a subject, comprising:
an electronic device having at least one processor and memory, wherein said processor is configured to execute instructions from a software application to cause the electronic device to perform the following step;
processing ion channel activity data for a plurality of predetermined voltage gated ion channels, wherein said ion channel activity data includes a measurement of the number of channels for each voltage gated ion channel, and the ionic flux for a particular ion for a plurality of voltage gated ion channels during a specified time period, and the specified time period of the ionic flux for a particular ion for each of the plurality of voltage gated ion channel; and
wherein the step of processing includes calculating an ion channel activity value for each of the plurality of voltage gated ion channels from the number of channels value and the rate of ionic flux value to produce quantified provided ion channel activity values.

12. The system for quantifying voltage gated channel activity for a subject of claim 11, additionally comprising an activity database having at least one quantified baseline ion channel activity value and embodied on non-volatile memory, wherein the processor is configured to additionally to cause the electronic device to interpret quantified provided ion channel activity values, wherein the step of interpreting includes comparing the quantified provided ion channel activity values with quantified baseline ion channel values.

13. The system for quantifying voltage gated channel activity for a subject of claim 12, wherein said quantified baseline ion channel activity values include theoretical ion channel activity values for at least one organ or cell type.

14. The system for quantifying voltage gated channel activity for a subject of claim 12, wherein said quantified baseline ion channel activity values include historical ion channel activity values.

15. The system for quantifying voltage gated channel activity for a subject of claim 12, wherein said quantified baseline ion channel activity values include known disease ion channel activity values for at least one organ or cell type.

16. The system for quantifying voltage gated channel activity for a subject of claim 15, wherein the step of interpreting additionally includes aiding in the diagnosis of membrane excitability disorders present by identifying known disease ion channel activity values which are substantially similar quantified provided ion channel activity values.

17. The system for quantifying voltage gated channel activity for a subject of claim 16, wherein the activity database additionally includes lifestyle factor data associated with ion channel activity and the diagnosing of membrane excitability disorders present includes a comparison of lifestyle factor data lifestyle factors relevant to the subject.

18. The system for quantifying voltage gated channel activity for a subject of claim 11, wherein the step of processing includes calculating ion channel activity in real time.

19. The system for quantifying voltage gated channel activity for a subject of claim 1, wherein the processor is configured to additionally to cause the electronic device to output ion channel activity, wherein the step of outputting includes providing a visual display of quantified provided ion channel activity values.

20. A method for quantifying voltage gated channel activity for a subject, comprising the steps of:
providing an electronic device having at least one processor configured to execute instructions from a software application;
providing ion channel activity data for a plurality of predetermined voltage gated ion channels, wherein said ion channel activity data defines measurements taken in a patient over a specified time period and includes a measurement of the number of channels for each voltage gated ion channel and measurements of the ionic flux for each voltage gated ion channel during the specified time period;
providing an activity database having at least one quantified baseline ion channel activity value, wherein said quantified baseline ion channel activity value includes at least known disease ion channel activity values for at least one organ or cell type;
configuring by said processor the ion channel activity data provided, wherein the step of configuring includes at least identifying the file format of the ion channel activity data provided and reading measurement values in the ion channel activity data provided;
processing by said processor the ion channel activity data provided, wherein the step of processing includes (1) calculating an ion channel activity value for each of the plurality of voltage gated ion channels from the number of channels measurement and the ionic flux measurements over the specified time period and (2) producing therefrom a quantified provided ion channel activity value for each voltage gated ion channel that is standardized for expression in electric current by time and voltage units, thereby enabling real time activity of the patient's voltage gated ion channels for the entire given excitable cell type to be summarized;
interpreting by said processor quantified provided ion channel activity values, wherein the step of interpreting includes comparing the quantified provided ion channel activity values with quantified baseline ion channel values and includes diagnosing membrane excitability disorders present by identifying known disease ion channel activity values which are substantially similar quantified provided ion channel activity values; and
outputting by said processor ion channel activity, wherein the step of outputting includes providing a visual display of quantified provided ion channel activity values.
